(19) United States
(12) Patent Application Publication
(10) Pub. No.: US 2024/0032820 A1
(43) Pub. Date: Feb. 1, 2024

(54) SYSTEM AND METHOD FOR SELF-LEARNING AND REFERENCE TUNING ACTIVITY MONITOR

(71) Applicant: MindMaze Group SA, Lausanne (CH)

(72) Inventors: Benoit MARIANI, Lausanne (CH); Farzin DADASHI, Lausanne (CH); Karim Kanoun, Lausanne (CH); Cleo Moulin, Lausanne (CH)

(21) Appl. No.: 18/162,081

(22) Filed: Jan. 31, 2023

Related U.S. Application Data
(63) Continuation of application No. 16/443,876, filed on Jun. 18, 2019, now abandoned.
(60) Provisional application No. 62/686,287, filed on Jun. 18, 2018.

(51) Int. Cl. A61B 5/11; A61B 5/00; G06V 40/20

(52) U.S. Cl. CPC A61B 5/1112; A61B 5/112; A61B 5/1118; A61B 5/681; G06V 40/25; A61B 2562/0219

(57) ABSTRACT

The present invention relates to systems and methods for self-learning of locomotion characteristic and speed during bipedal locomotion using sensor and GPS technology.

The present invention relates to systems and methods for self-learning of gait characteristic and speed during bipedal locomotion using sensor and GPS technology.

### BACKGROUND

Mitigating the risk of falls is a primary medical concern for the elderly. Predicting falls is one method for mitigating the risk. Scientific evidence suggests that increased gait variability is a key prediction factor of falls among community-living older adults. Hausdorff et al., 2001. Also, the 5-year survival rate of patients above 65 years old can be predicted from gait speed along with age and gender. Studenski et al., 2011. Gait speed is also a good predictor of future risks of falls and hospitalizations. Studenski et al., 2003. Studies have also provided normative gait speed values such as the sufficient speed to cross the street of 1.22 m/s [Langlois et al., 1997] and the comfortable speed of various patients [Bohannon et al, 1997].

The science of predicting falls currently relies on many different factors including past experience (e.g., frequency, context, and severity) with falls, visual impairment, cognitive impairment, and psychology (e.g., depression in the patient), to name a few. Gait analysis is one consideration. Typically, all of these factors are combined to assign a risk of fall category to the elderly. Living community staff (i.e., therapists, nurses, doctors, and the like) can monitor each patient in the community based on the risk category. Stalenhoef et al., “A Risk Model for the Prediction of Recurrent Falls in Community-Dwelling Elderly: a Prospective Cohort Study,” J Clin Epidemiol, 2000.

However, insurance providers and clinical institutions struggle to meet the challenges of caring for and treating the elderly at risk of falls and the fallen and injured elderly. The growth rate in the number of elderly and strict regulations result in fewer doctors and other caregivers and, thus, contribute to the challenges. Additionally, the ageing population, as wells as their families, wish to continue living at home for as long as possible but mobility limitation and falls prevent them from doing so.

However, current systems and methods have only small to moderate effects on gait deficits and focus on treating patients after adverse events when it is too late. This is likely due to a lack of appropriate tools available to the health care providers to understand their patients' behavior and physical abilities post-consultation. The current systems and methods do not meet clinicians' needs for an easy yet reliable functional evaluation of patient status. The current systems and methods are thus unable to provide objective evaluation, prevent falls, and prescribe interventions which would benefit patients. Indeed, current systems generally provide gait measurements specifically for clinical evaluation and assessed in laboratory settings in protocoled conditions. These time-consuming evaluations are thus limited to a very restricted number of patients.

To compensate, some clinicians have turned to basic trackers found on the market, such as pedometers, to understand their patients' behavior in out-of-the-lab conditions. As discussed in more detail below, these tools are not able to provide them with the relevant parameters, such as speed for example, with sufficient accuracy to be meaningful.

The state of the art includes a wide range of movement measuring devices and methods, as discussed below. Generally, these devices and methods either indicate that a fall is underway or do not provide reliable data and, consequently, lack market acceptance past short-term use. Indeed, a report by PricewaterhouseCoopers showed that the average use of FitBit is two weeks due to limitations of reliability. This reports also states that “[c]onsumers recognize enormous potential in the emerging category—but right now, they are skeptical that wearable technology can deliver on that potential.” (http://www.mobihealthnews.com/37543/pwc-1-in-5-americans-owns-a-wearable-1-in-10-wears-them-daily).

For example, Petelenz, U.S. Pat. No. 6,433,690, describes a method for detecting whether a movement is a fall by recording the acceleration and body position data using an accelerometer. Petelenz attempts to distinguish a fall from other accelerated movements and determine the severity of the fall for the purpose of sending an alert signal. Thus, Petelenz is useful only for indicating a fall has already occurred.

Some existing systems provide long-term gait analysis by providing floor-based sensing. For example, Alwan, U.S. Pat. No. 8,894,576 describes a floor-based sensing system to gather longitudinal data of a person's gait to use in a fall prediction model. Alwan describes a prediction model based on distinguishing normal and abnormal gait. The gait characteristics include step count, pace, normal condition, limp shuffle, falls, average walking velocity, step or stride length. Such systems can be effective but require the subject to walk on the monitored surface and are ineffective elsewhere. Furthermore, such systems can be expensive, requiring specialized flooring systems and may not be easily replaced or repaired.

Azzaro, U.S. Pat. No. 7,612,681, describes collecting range-controlled radar sensor data an applying different techniques to predict the fall risk likelihood by distinguishing among a normal walk, a limp, or a shuffle using a wavelet analysis. Techniques include a Hidden Markov Model and a Bayesian network. Azzora requires potentially expensive motion sensing equipment throughout the living quarters of the subject. Additionally, data is captured only where motion sensing equipment is lacking or outside the living quarters. Thus, systems like Azzaro fail to capture relevant data, including gait data captured in areas less familiar to, and thus riskier for, the subject.

Some existing systems attempt to predict whether a fall is imminent. For example, Kasama, U.S. Pat. No. 9,299,235 describes detecting acceleration in the gravitational acceleration direction of the portable electronic apparatus, determining whether or not the acceleration in the gravitational acceleration direction is a threshold value or less, the threshold value being stored in a determination threshold value table, and raising an alarm for prediction of stumbling of a user when the acceleration in the gravitational acceleration direction is the threshold value or less. Kasama collects three-dimensional acceleration data and calculates step frequency to determine an immediate risk of falling. Kasama does not, however, assist in determining a generalized risk of falling and focus solely on whether a fall is imminent or in progress.

What is needed is a long-term monitoring system useful in generating a generalized risk of falls along with a determination of imminent fall risk so that the risk can be managed before a fall and proper therapy can be applied after a fall. Additionally, there is a need for systems and methods that allow remote caregivers, friends, and loved ones to be involved in fall prevention of the elderly.

### SUMMARY OF SOME OF THE EMBODIMENTS

Aspects of the invention relate to deriving a generalized risk determinant using low-cost sensors and GPS to accurately measure gait speed.

In one embodiment, advanced gait parameters are measured from global distance and position metrics and detailed kinematics metrics and received by a third-party insurance provider. A discount tier is determined by the third-party insurance provider from the advanced gait, or more generally, locomotion, parameters and customer threshold requirements. In another embodiment, advanced locomotion parameters are measured from global distance and position metrics and detailed kinematics metrics and received by a clinical system outside of a protocolled condition context. The clinical system can generate a gait or locomotion analysis summary. In another embodiment, advanced locomotion parameters are measured from global distance and position metrics and detailed kinematics metrics and received by a remote server. A risk determinant can be generated from the advanced locomotion parameters and a message signal based on the risk determinant can be sent from the remote server.

Unless otherwise defined, all technical and scientific terms used herein have the same meaning as commonly understood by one of ordinary skill in the art to which this disclosure belongs. The materials, methods, and examples provided herein are illustrative only and not intended to be limiting.

Various embodiments of the methods, systems and apparatuses of the present disclosure can be implemented by hardware and/or by software or a combination thereof. For example, as hardware, selected steps of methodology according to some embodiments can be implemented as a chip and/or a circuit. As software, selected steps of the methodology (e.g., according to some embodiments of the disclosure) can be implemented as a plurality of software instructions being executed by a computer (e.g., using any suitable operating system). Accordingly, in some embodiments, selected steps of methods, systems and/or apparatuses of the present disclosure can be performed by a processor (e.g., executing an application and/or a plurality of instructions).

Although embodiments of the present disclosure are described with regard to a “computer,” and/or with respect to a “computer network,” it should be noted that optionally any device featuring a processor and the ability to execute one or more instructions is within the scope of the disclosure, such as may be referred to herein as simply a computer or a computational device and which includes (but not limited to) any type of personal computer (PC), a server, a cellular telephone, an IP telephone, a smartphone or other type of mobile computational device, a PDA (personal digital assistant), a thin client, a smartwatch, head mounted display or other wearable that is able to communicate wired or wirelessly with a local or remote device. To this end, any two or more of such devices in communication with each other may comprise a “computer network.”

### BRIEF DESCRIPTION OF THE DRAWINGS

Embodiments of the disclosure are herein described, by way of example only, with reference to the accompanying drawings. With specific reference now to the drawings in detail, it is stressed that particulars shown are by way of example and for purposes of illustrative discussion of the various embodiments of the present disclosure only and are presented in order to provide what is believed to be a useful and readily understood description of the principles and conceptual aspects of the various embodiments of inventions disclosed therein.

FIGS. 1-3 illustrate schematics of exemplary systems for classifying a locomotion kinematics sequence, estimating speed, and self-learning of a user's locomotion characteristics for monitoring gait deterioration in accordance with preferred embodiments.

FIG. 4 illustrates a flowchart for an exemplary method for classifying a locomotion kinematics sequence, estimating speed, and self-learning of a user's locomotion characteristics for monitoring gait deterioration in accordance with preferred embodiments.

FIG. 5 illustrates a flowchart of an exemplary method for classifying a locomotion kinematics sequence and estimating speed of a user's gait in accordance with preferred embodiments.

FIG. 6 illustrates a flowchart of an exemplary method for classifying a locomotion kinematics sequence for monitoring gait deterioration in accordance with preferred embodiments.

FIG. 7 illustrates an exemplary locomotion kinematics sequence representation for walking derived from a wrist-worn sensor device in accordance with preferred embodiments.

FIG. 8 illustrates an exemplary locomotion kinematics sequence representation for running derived from a wrist-worn sensor device in accordance with preferred embodiments.

FIGS. 9-10 illustrate exemplary representations of matching of a locomotion kinematics sequence to a reference model template with and without dynamic time warping, respectively, in accordance with preferred embodiments.

FIG. 11 illustrates an exemplary visualization of the results on a smartwatch platform. Movement context (indoor vs. outdoor), number of steps, cadence, speed of locomotion and displacement with(out) GPS, auto-classification of activity, barcode of daily physical activity (PA) and complexity of PA are among preliminary information that can be displayed.

FIG. 12 illustrates a schematic of an exemplary system including a reference model and template and modules for generating data, classifying data, and updating the reference model based on the data.

### DETAILED DESCRIPTION OF SOME OF THE EMBODIMENTS

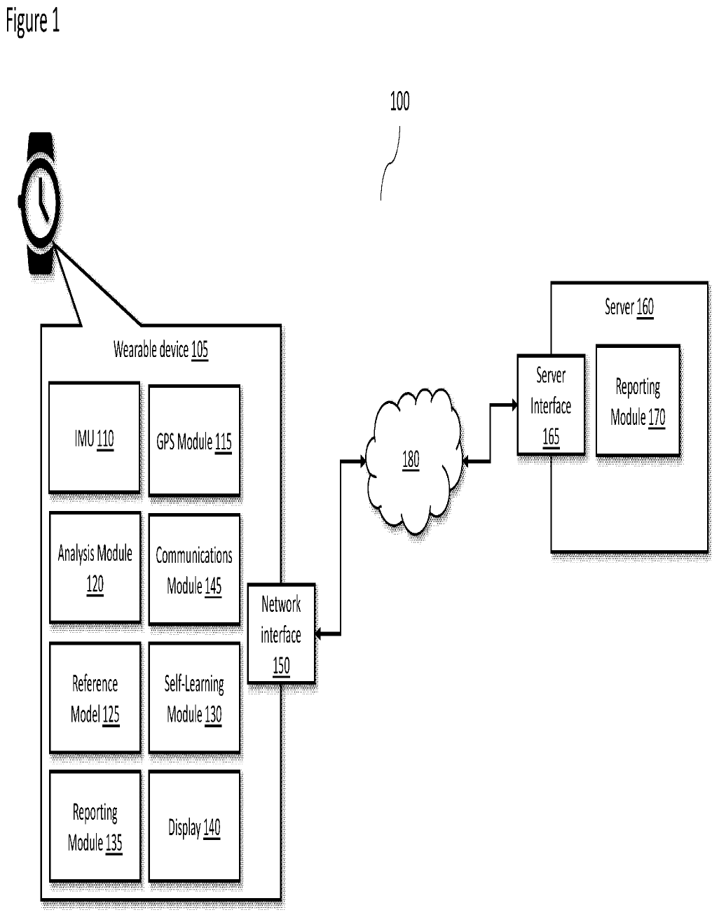

Referring now to FIG. 1, a schematic is illustrated for an exemplary system for self-learning tracking of gait speed. System 100 features a user device 105. In preferred embodiments, user device 105 can be a wrist-worn device, similar to a watch, smartwatch, or fitness band. The user is assumed to be holding, wearing, or otherwise be attached to user device 105, such that movements of the user are reflected in movements of user device 105. Preferably, user device 105 is wrist-worn so that users can easily put it on or remove it as necessary and so that gait or locomotion movements are reflected in movements of the device. In other embodiments, user device 105 can be a mobile communications device, such as a cellular telephone for example, or other portable computing device. User device 105 is preferably able to perform most, if not all, of the analysis functions independently. User device 105 is in communication with one or more remote servers 160 through a computer network 180, as shown, to provide for additional services or to access, store, or share data. Computer network 180 may optionally comprise the Internet or an internal wired or wireless network.

User device 105 features an IMU (inertial measurement unit) 110 for collecting angular velocity and linear acceleration data, in regard to movements of user device 105, thereby collecting such data about movements of the user. IMU 110 is preferably selected for a suitable sensitivity and range, according to the functions described in greater detail below. User device 105 can also feature a barometric pressure sensor. The barometer is used to enrich movement context and improve the accuracy of energy expenditure estimation. User device 105 also comprises a GPS or other type of positioning system module 115 for collecting positioning data, in regard to movements of user device 105, thereby collecting such data about movements of the user. GPS module 115 is preferably selected for a suitable sensitivity and range, according to the functions described in greater detail below and can include one or more positioning data receivers. GPS module 115 provides geolocalization to compute with the highest precision gait speed and walking distance. Besides, GPS data is used as the reference training data to build up a data-driven model for gait speed and walking distance in indoor areas. Once the data driven model is built, GPS usage can be temporized to enhance the wearable device battery life. It should be understood as implied above that other positioning systems can be used in addition to or in place of GPS and that the use of “GPS” herein can refer to the use of one or more such satellite-based navigation systems. In addition, those of skill in the art can appreciate that other types of positioning systems such as local position systems that generally use a type of signaling beacon to determine a position can be used in addition to or in place of a satellite-based navigation system. GPS module 115 can include a receiver for communicating with the appropriate satellite or beacon and other hardware and/or software suitable to allow for the collection of positioning data.

Within the user device 105, optionally the following components are included as a non-limiting implementation example:
Input 3D accelerometer @ 100 Hz
Input 3D gyroscope @ 100 Hz
Input 3D magnetometer @ 50 Hz
Embedded C library with minimal footprint (12 kb allocated memory for execution on Nucleo F4 or other processing platform with similar or greater specifications)

An analysis module 120 receives such data from IMU 110 and then classifies the locomotion of the user according to such data and one or more reference model templates. As described in greater detail below, such activity classification preferably features determining a gait, or locomotion, category, such as walking, jogging, running, climbing stairs, walking with a cane, and the like. Activity classification can be performed as described in PCT Application No. PCT/IB2018/059933, which is hereby incorporated by reference as if fully set forth herein.

Self-learning module 130 receives data from IMU 110 and GPS module 115 to update one or more reference model templates in reference model 125 according to the data. User device 105 also includes communications module 145 for sending data to one or more servers 160 for further processing, including reporting functions. The speed calculation model is updated during outdoor activities or activities in which the speed of the user is more readily determined, such as when the user is using a connected treadmill for example. As discussed in further detail below, if GPS data is available, user device 105 can provide more accurate speed and locomotion kinematics data that if only IMU data is available. Furthermore, relevant reference model templates can be updated to more closely match the gait of the user with GPS data, if available. The algorithmic intelligence to update reference model templates is based on advanced machine learning techniques to improve indoor tracking by fusing IMU data with GPS data during outdoor measurements. Analysis module 120, self-learning module 130, and communications module 145 and other modules of device 105 can be further separated into other modules or combined. Furthermore, the modules can be implemented either in software or hardware.

Reference model 125 preferably includes one or more reference model templates representing different types of gaits. In some preferred embodiments, reference model templates can be created from locomotion kinematics sequence (e.g., as a time series) data obtained from one or more people preferably having a cross-section of locomotion characteristics (e.g., gait characteristics). In other embodiments, reference model templates can be created as a synthetic template in which the template is estimated from known or common locomotion characteristics. In other preferred embodiments, reference model can be built over time and include data from the user. In this case, locomotion kinematics sequence data from the user can be collected as part of a calibration process to create a reference model template. Such a calibration process would be a supervised process to ensure accurate classification of locomotion kinematics sequence data and template creation. In some instances, a reference model template can be customized to the gender, age, or physical characteristics of different types of users. Physical characteristics can include height, leg height, weight, and the like. Thus, a reference model template can be matched to a user based on one or more of these characteristics for analysis as described further herein. Reference model 125 can be stored according any well-known data storage or memory technique or device. In preferred embodiments, reference model can include data from one or more anonymous users or a synthetic model. In some instances, reference model can be stored at server 160 and at wearable device 105 and be updated periodically if changes are made to one or the other.

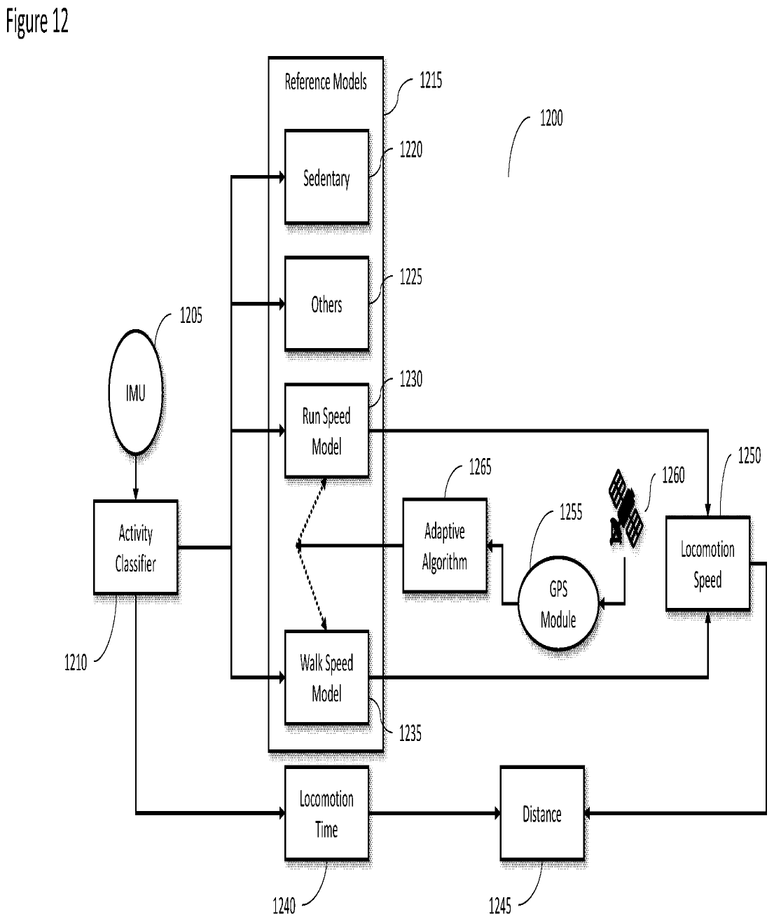

Self-learning module 130 can include hardware or software for updating reference model 125 from data sampled from GPS module 115, IMU 110, or both (see, e.g., the block diagram of FIG. 12). Preferably, self-learning module 130 is configured to receive, from analysis module 120, speed and locomotion kinematics sequence data derived from both IMU 110 and GPS module 115 and configured to apply one or more machine learning techniques to update one or more relevant reference model templates in reference model 125.

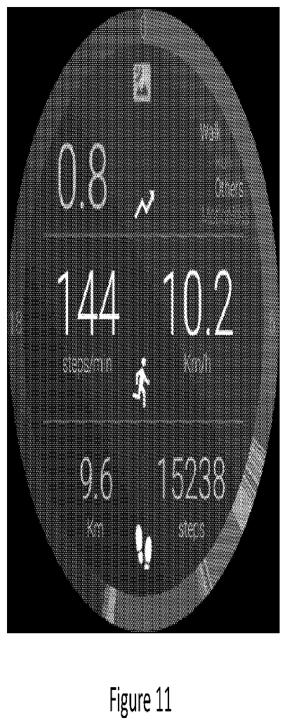

The results of the classification may be displayed on a display 140 of the wearable device 105, which may be integrally formed with or attached to wearable device 105. For example, FIG. 11, discussed further below, illustrates an exemplary display. Wearable device 102 also preferably features a user interface, which is displayed on display 140. Preferably, the results of the classification, as well as other visual information, is displayed through user interface by display 140. A user interface also preferably accepts user commands, as display 140 is preferably a touchscreen. For example, the user may optionally select which data is to be displayed and for which time period. In some embodiments, other types of user interfaces can be used, including an audio user interface in which classification, other results or information, user messages, and the like can be presented. In some cases, an audio user interface can present menu options and accept commands. In this way, wearable device 105 can be configured for more accessible use.

Communications module 145 is coupled to a network interface 150 to communicate with server 160 over network 180. Data from the various modules of wearable device 105 or IMU 105 or GPS module 110 may be shared with server 160 through such communication. Server 160 includes a network interface 165 for communications and reporting module 170. Reporting module 170 can be hardware or software and can be further combined with or separated from other components of server 160. Reporting module 170 can generate reporting information about the movements of the user including gait speed over time, average gait speed over a time, locomotion classification, visual representations of locomotion kinematics, and the like. System 100 can include other remote servers that provide reporting services or other processing or data storage services. In some other embodiments, wearable device 105 or other type of user device (e.g., smartphone, tablet, and the like) can include a reporting module, a display for displaying reporting information, or both.

Wearable device 105 is preferably able to perform most, if not all, of the analysis functions independently. However, wearable device 105 is preferably in communication with a remote server 160 through a computer network 180 as shown, for additional services, and optionally to access and/or share additional data. Computer network 180 may optionally comprise the Internet for example. The user is assumed to be holding, wearing or otherwise to be attached to wearable device 105, such that movements of the user are reflected in movements of wearable device 105.

It should be understood that each of the modules and components described above can be combined into fewer modules, components, or devices or further separated into additional modules, components, or devices to perform the functions described herein in a more consolidated or more distributed manner. Furthermore, wearable device can include one or more memory storage device that contains one or more of the modules or computer processor instructions that implement a portion or all of the modules.

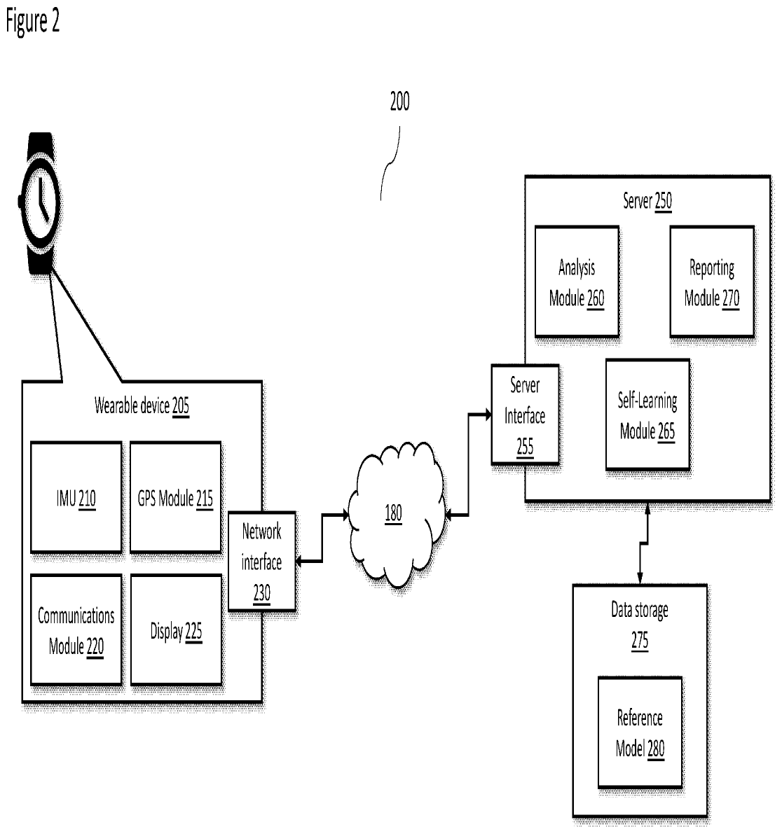

Referring now to FIG. 2, a schematic of a system 200, featuring a wearable device 205, is shown. In this example, wearable device 205 can be similar to wearable device 105. However, wearable device 205 does not include an analysis module, self-learning module, or a reference model. Instead, server 250 includes an analysis module 260, self-learning module 265, and reporting module 270. In communication with server 250 is a data storage device 275 for reference model 280. Thus, in embodiments consistent with the schematic of FIG. 2, IMU and GPS data can be sent to server 250 via wired or wireless communication over network 180 and server interface 255.

The remaining components of wearable device 205 are similar to the other components of wearable device 105, including IMU 210, GPS module 215, communications module 220, display 225, and network interface 230. In preferred embodiments in accordance with system 200, communications module is configured to send data from IMU 210 and GPS module 215 to server 250 for additional processing.

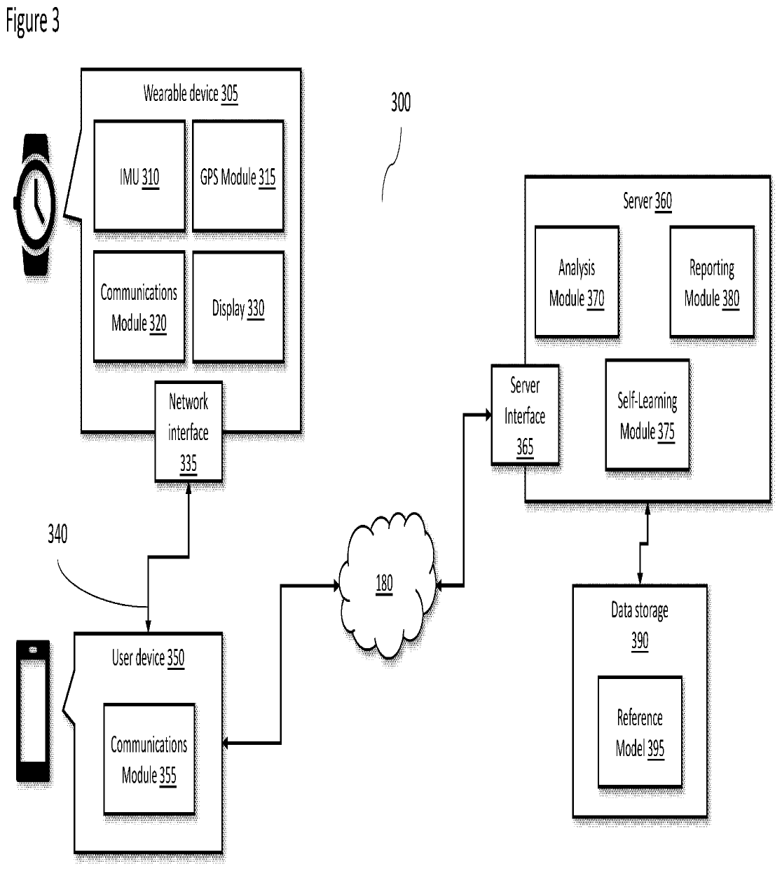

FIG. 3 illustrates an exemplary alternative configuration 300 of the systems illustrated in FIGS. 1 and 2, in which various functions performed by the wearable device 105 or wearable device 205 are instead performed by another user device 350. User device 350 may optionally comprise a smartphone, tablet, PC, or other computing device that can be in communication with wearable device 305. User device 350 can be in communication with wearable device 305 through a wired or wireless connection 340.

In the example shown, user device 350 includes communications module 355 to transmit data, receive data, or both on behalf of wearable device 305 to and from server 360 via network 180. User device 350 can be configured to provide other services, including but not limited to displaying data or reporting information or to analysis and processing services described herein. For example, in some preferred embodiments, user device 350 can include an analysis module, self-learning module, reporting module, reference model, or some combination thereof to provide analysis or services. IMU 310, GPS module 315, communications module 320, display 330, and network interface 335 in wearable device 305 substantially correspond to the IMU 110 and 210, GPS module 115 and 215, communications module 145 and 220, display 140 and 225, and network interface 150 and 230 from FIGS. 1 and 2, respectively. Furthermore, analysis module 370, reporting module 380, self-learning module 375, and server interface 365 substantially correspond to analysis module 120 and 260, reporting module 135, 170, and 270, self-learning module 130 and 265, and server interface 165 and 255 of FIGS. 1 and 2. Lastly, data storage 390 and reference model 395 substantially correspond to data storage 275 and reference model 135 and 280 of FIGS. 1 and 2.

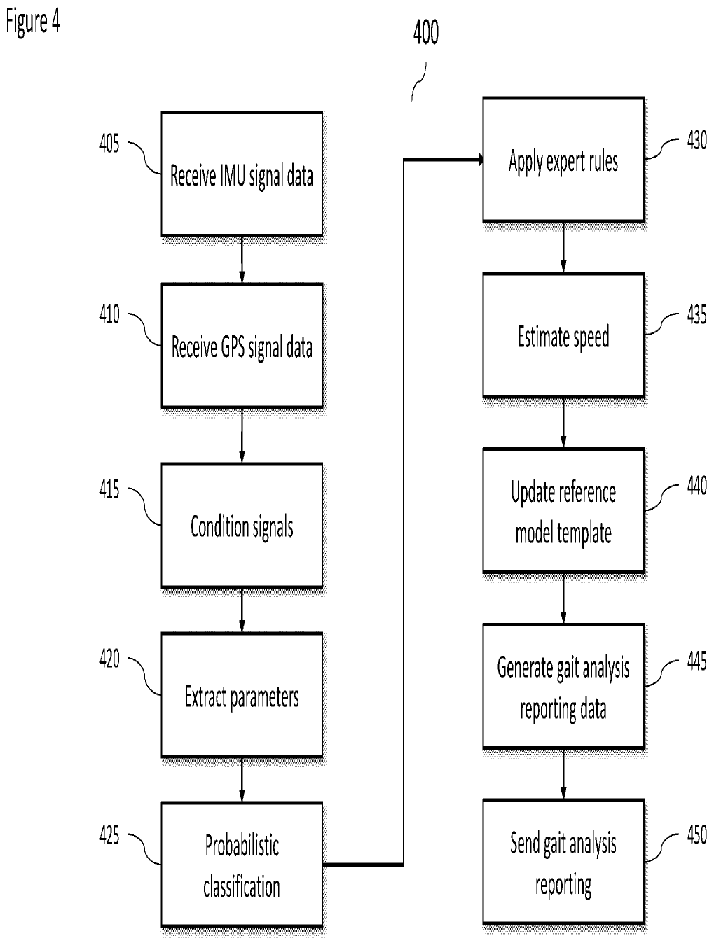

Referring now to FIG. 4, a flowchart of an exemplary method 400 for tracking locomotion characteristics, classifying a locomotion kinematics sequence, and estimating speed using a reference model is illustrated. The method may be performed with embodiments similar to the systems of FIGS. 1-3 or other systems. At step 405, an IMU signal is received. The IMU signal preferably is sampled from a device such as device 105. In preferred embodiments, the IMU signal includes time series IMU data or is converted to a data structure holding a time series, for example, as the data is sampled with the timestamping corresponding to the sampling rate. At step 410, GPS signal data is received. In accordance with preferred embodiments, the GPS signal data includes time series GPS position data or, to the extent time is not included in the positioning data, is converted to a data structure holding a time series, for example, as the data is sampled with the timestamping corresponding to the sampling rate. Furthermore, in preferred embodiments, the user device includes a GPS module from which the GPS data is sampled. In other embodiments, it is possible to have a GPS-enabled device that communicates with user device or that is in communication with a server to receive the GPS data.

At step 415, the IMU and GPS signals are conditioned. Such signal conditioning preferably includes performing a dynamic calibration so IMU axes are virtually aligned to the functional movement axes. The calibration is preferably performed as an optimization that minimizes the difference between virtually-rotated-IMU signals and the function axis of body segments. Such a calibration means that the analyzer is able to determine the activity parameters without requiring specific direction of attachment of IMU to the user body.

At step 420, parameters are extracted from IMU signals. Extracted parameters have or reveal biomechanical information on the user's gait which can include duration of movement, velocity, and IMU orientation in 3D space (e.g., position along different axes). Optionally, statistical features are extracted cycle by cycle. The feature extraction is insusceptible to cycle duration or amplitude and mainly dependent on geometric shape of the IMU signal at each cycle.

At step 425, probabilistic classification is performed. In preferred embodiments, a label that indicates the type of activity and a confidence interval on the certainty of chosen label is determined for the locomotion kinematics sequence data. Once the IMU is aligned to the bodily axis through, for example, signal conditioning as discussed above, and movement generic parameters are extracted, then the activity type or locomotion can be classified. In preferred embodiments, activity or locomotion classification is limited to particular types of gait involving legged locomotion of the user. Legged locomotion of the user can include, in some cases, locomotion with assistance (e.g., walking with a cane or walker). It is possible for some embodiments to classify other types of locomotion. IMU signals that indicate some movement other than locomotion are disregarded. Optionally dynamic time warping is applied to the data, to account for temporal effects. For example, the classification may be performed according to multi-class QDA (quadratic discriminant analysis), a technique which is well known in the art. The features used for the covariance matrix of the QDA preferably include, but are not limited to, statistical features such as signal amplitude, auto regressive coefficients that describe each cycle of IMU data (preferably in 6 channels), and signal form features extracted from the dynamic time warping.

At step 430, expert rules are preferably applied, based on the output of probabilistic classifier and temporal sequence of activity in terms of previous activities. According to the application of such rules, the activity type is preferably accepted or modified.

At step 435, a speed is estimated. Preferred embodiments can use a Locally Linear Model Tree (LoLiMoT) speed estimator updater. A LoLiMoT is a subset of fuzzy algorithms for input space decomposition with local linear least squares optimization. The input space is decomposed in an axis-orthogonal manner yielding hyper-rectangles which accommodate fuzzy membership functions. The standard deviation of membership functions is chosen proportionally to the extension of hyper-rectangle. LoLiMoT provides a flexible framework to model intrinsic nonlinearities between input and output spaces. LoLiMoT was used to find the model that maps Kinematics features to running (walking) speed. Other preferred methods can estimate speed based on sensor data. For example, in some embodiments, it is possible to bypass a speed estimation from IMU data and use only the GPS data.

At step 440, the relevant reference model template determined and updated. If reference template data is available, the LoLiMoT estimator, updates the mathematical speed model of the user which is preferably part of a reference model as discussed in connection with FIGS. 1-3.

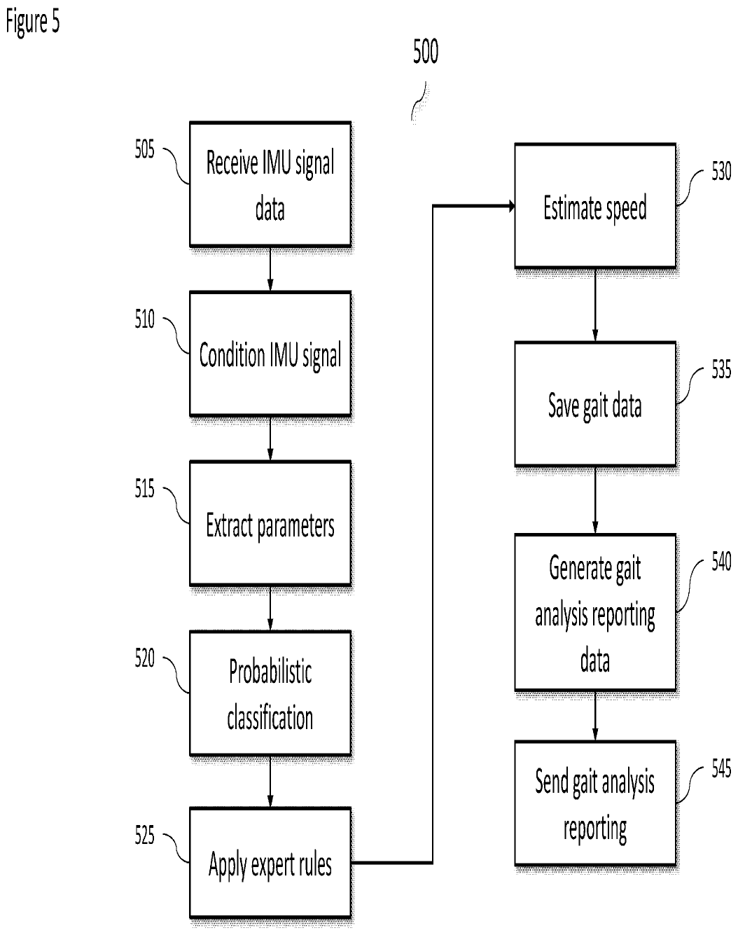

FIG. 5 relates to methods applied if the user is in the areas where GPS (reference) data is not available or the GPS module is powered off.

At step 445, locomotion characteristic data for reporting are stored in a gait reporting data store. In some preferred embodiments, a user device can have a data storage or memory component in which the data classification and speed. In some preferred embodiments, locomotion characteristic data is stored on the user device temporarily and periodically or on-command sent to another server for storage and further analysis and/or processing. Locomotion characteristics can include steps, cadence, speed, duration, number of sessions, and the like, as well as metadata associated with the characteristics such as time, date, and location, if available.

At step 450, gait analysis report is created and sent. In some preferred embodiments, reporting is sent to a caregiver (e.g., doctor, therapist, nurse) to diagnose gait problems or to monitor gait change trends in the user. Reporting can take the form of any well-known reporting technique including printing, email, web page, and the like. Reporting can also be sent or made available to the user or others. In some preferred embodiments, reporting can be sent to a user device such as user device 105.

Referring now to FIG. 5, a flowchart for an exemplary method 500 for analyzing and reporting gait according to preferred embodiments. Method 500 is similar to method 400 except that a GPS signal is unavailable. Thus, in preferred embodiments, a reference model template is not updated. At step 505, an IMU signal is received, similar to step 405. At step 510, the IMU signal is conditioned. At step 515, parameters are extracted from IMU signals. At step 520, activity or locomotion classification is performed or at least a portion of activity or locomotion classification is performed. At step 525, expert rules are applied based on the output of probabilistic classification and temporal locomotion kinematics sequence in terms of previous activities. According to the application of such rules, the activity type is preferably accepted or modified. At step 530, a speed is estimated. At step 535, the locomotion kinematics sequence and speed data are saved.

At step 540, gait analysis reporting data is generated. Such data can include temporal locomotion kinematics sequence data, speed data, and the like. Reporting data generated in preferred embodiments is discussed in further detail above. At step 545, gait analysis reporting is sent. This step can be performed in response to a request, for example, as a request for a web page or the presentation of a reporting user interface. In some instances, this step can be performed in response to a periodic or event-based trigger. For, example, periodic reporting data can be sent as an email or through some other messaging or user interface presentation or in response to a threshold related to a user's gait deterioration or development.

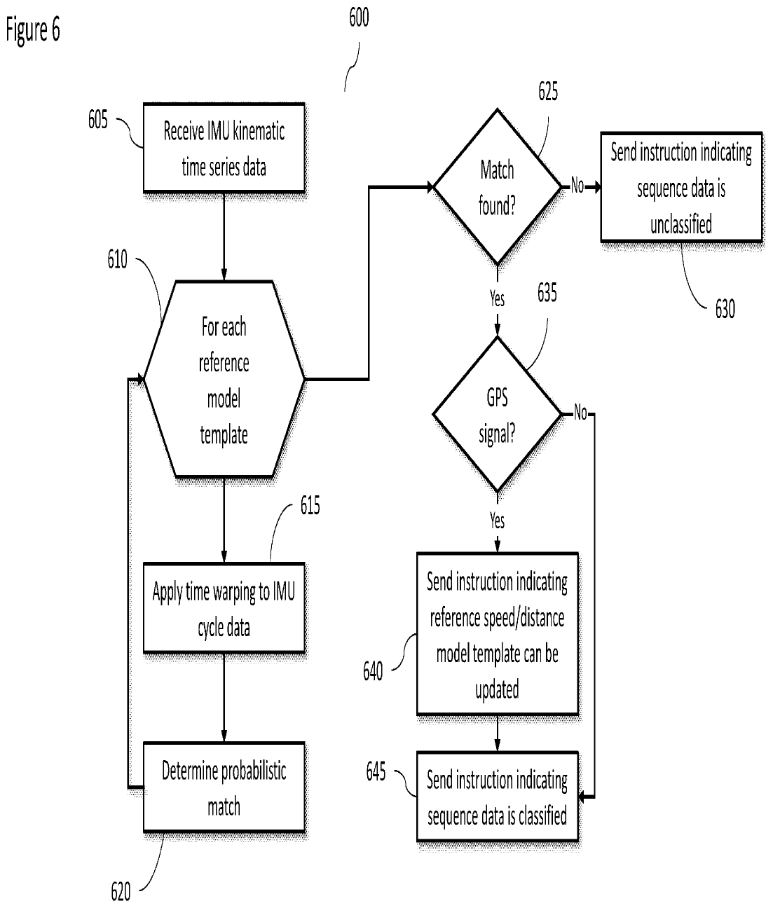

FIG. 6 illustrates a flowchart of an exemplary method 600 of probabilistic classification of a user's locomotion. In preferred embodiments, method 600 corresponds to step 425 for probabilistic classification from FIG. 4. At step 605, the time-series data that represents kinematics of activity is received from IMU. In preferred embodiments, time data is combined with raw or processed IMU to generate time series data.

At step 610, a reference model template is received and a probabilistic match to the IMU locomotion kinematics sequence data is determined. This step and steps 615 and 620 are repeated for each reference model template. In some preferred embodiments, only relevant reference model templates can be used. A relevant reference model template can be based on criteria such as speed range or templates selected as relevant to the user. At step 615, dynamic time warping is applied to the IMU cycle data. As described herein, for the probabilistic match, some technique known in the art, for example QDA, is used. In preferred embodiments, a confidence level for the probabilistic match is generated. In preferred embodiments, a confidence level or margin of error range preferably is from 10-20% and more preferably 15%. If a match is not found, then at step 630, an instruction is sent indicating that sequence data is unclassified. In this case, in some preferred embodiments, the locomotion kinematics sequence data can still be saved for reporting and the reporting can indicate that is was unclassified. In some preferred embodiments, the locomotion kinematics or activity sequence data can be classified as provisional based on the closest matching reference model template. Later, the locomotion kinematics or activity sequence data can be corrected for noise or a reference model template can be updated such that the locomotion kinematics or activity sequence data matches within the threshold and, thus, be classified.

If a match is found for the locomotion kinematics or activity sequence data, then at step 635, a check is made whether GPS signal data is available. If so, then at step 640, an instruction is sent indicating that the relevant reference model template can be updated and at step 645, an instruction is sent indicating that the locomotion kinematics or activity sequence data can be classified. If GPS signal data is not available, then step 645 is performed, but step 640 is not. The instructions sent can be flags stored in a buffer that are communicated with locomotion kinematics or activity sequence or other data to a segment of an analysis module self-learning module, communications module as described herein or some other module for processing locomotion kinematics or activity sequence data. For example, in an embodiment consistent with system 100, an instruction flag can be received by communications module 145 in preparation for sending data to server 160 or by self-learning module 130 in preparation for updating or skipping an update of a reference module template in reference model 125.

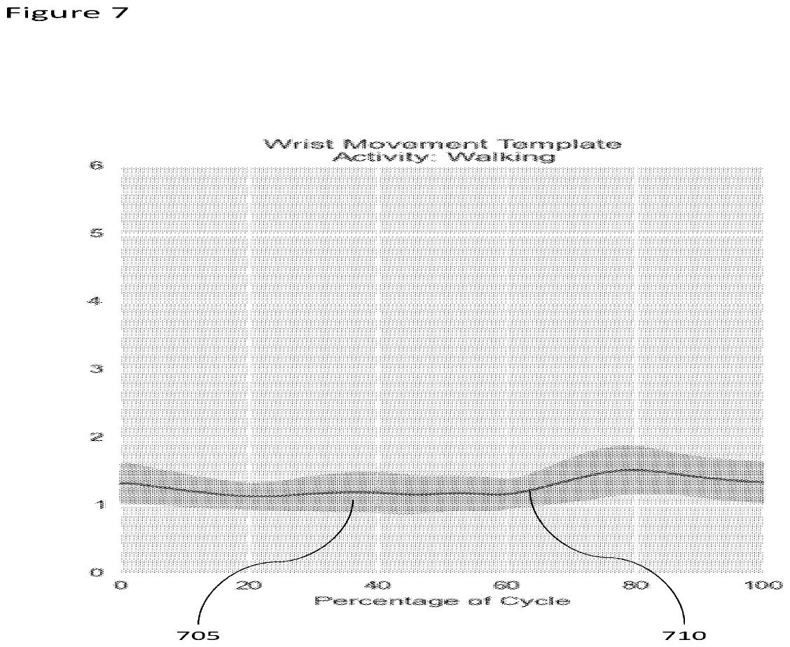

FIG. 7 illustrates an exemplary time series graph representing a walking kinematics template model. Along the x-axis of the graph, the time is represented as a percentage of the time of the entire cycle. The y-axis represents the amplitude of acceleration along a vertical axis. The shaded area 705 represents a margin of vertical acceleration variability such that where the measured locomotion kinematics sequence data line 710 falls within the shaded area entirely, the locomotion can be classified as walking. Other preferred embodiments can include a reference model of other or additional data points, including frontal and medio-lateral acceleration and angular velocity from wrist or other sites of the body.

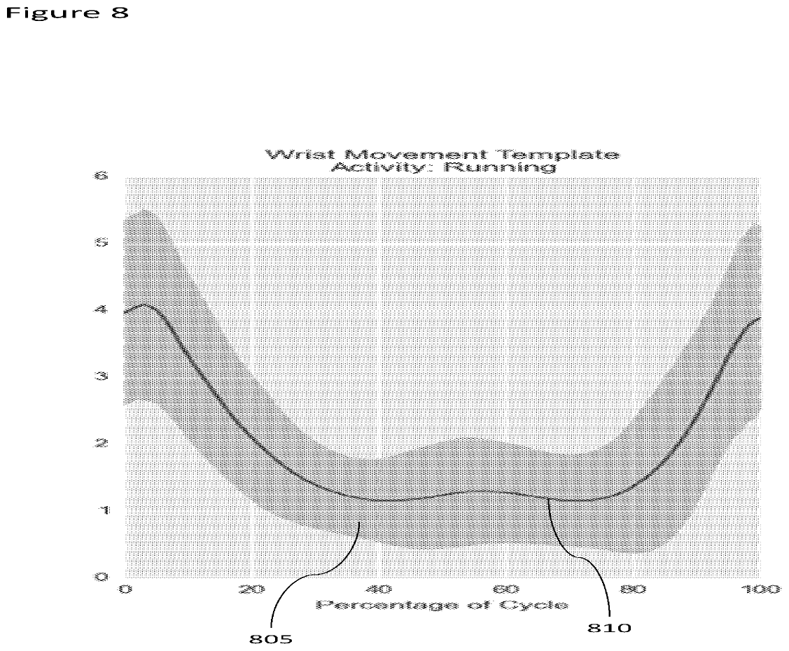

FIG. 8 illustrates another exemplary time series graph representing a running-cycle data from IMU. As can be seen, the y-axis acceleration of the user device is more varied than in the time series graph representing a walking gait, or locomotion, classification from FIG. 7. The shaded area 805 again represents the margin of variability and line 810 represents average measured sequence data.

Once locomotion type has been identified, the IMU-based speed calculation model, corresponding to the locomotion, can be updated based on reference GPS data. In fact, this scheme is capable of personalizing speed estimation based on kinematics of user.

In preferred embodiments, the reference model includes templates for walking and running as discussed in connection with FIGS. 7 and 8 as well as templates for other gait, or locomotion, classifications. For example, a template can exist for gait, or locomotion, classifications between walking and running such as a jog.

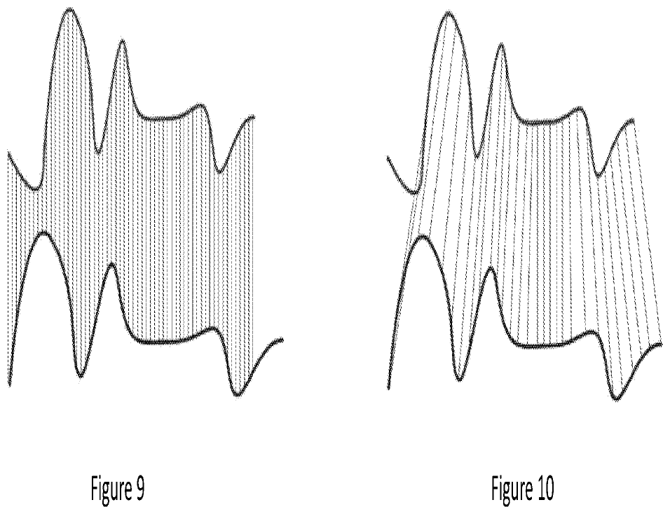

FIG. 9 illustrates a schematic of an exemplary comparison of actual data with a template according to preferred embodiments. The top line represents a template time series and the bottom line represents an actual recorded data time series. As illustrated in FIG. 10, in preferred embodiments, dynamic time warping is applied to the recorded data to fit the recorded data to the template. Dynamic time warping corrects for errors and noise in the data. Additionally, dynamic time warping corrects for minor changes in acceleration by the user that may not be consistent across every stride.

Referring now to FIG. 12, a block diagram schematic of a system 1200 with a reference model updated using IMU and GPS data is illustrated. System 1200 includes an IMU 1205 and GPS module 1255. GPS module 1255 is in communication with GPS satellite 1260. Activity classifier 1210 receives data generated by IMU 1205. In some embodiments, activity classifier can process IMU data as described herein, including conditioning, time stamping, and the like. Activity classifier 1210 classifies locomotion as described above as one of the activities in reference model 1215, which can include templates for sedentary 1220, run speed 1230, walk speed 1235, and other types of activity or locomotion templates 1225. For locomotion classified as run or walk the relevant locomotion speed 1250 can be combined with locomotion time 1240 generated by activity classifier 1210 to generate distance data 1245.

System 1200 also includes GPS module 1255 which generates GPS data as described herein. GPS data is received by adaptive algorithm 1265 to adjust a locomotion reference model template. In system 1200, one of run speed model 1230 or walk speed model 1235 can be updated based on criteria as described herein. In other embodiments, additional models can be included and can be updated. Locomotion time 1240, distance 1245, locomotion speed 1250, and GPS data can be stored for later retrieval for reporting or other purposes.

Any and all references to publications or other documents, including but not limited to, patents, patent applications, articles, webpages, books, etc., presented in the present application, are herein incorporated by reference in their entirety.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and apparatuses which may further include any and all elements from any other disclosed methods, systems, and apparatuses, including any and all elements corresponding to target particle separation, focusing/concentration. In other words, elements from one or another disclosed embodiment may be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patentably distinct from one and/or another reference by specifically lacking one or more elements/features. In other words, claims to certain embodiments may contain negative limitation to specifically exclude one or more elements/features resulting in embodiments which are patentably distinct from the prior art which include such features/elements.

What is claimed is:

1. A method for monitoring gait analysis for a person, comprising:
sampling inertial data from an IMU sensor configured to be mounted to the person while performing an activity categorized according to a locomotion category;
generating a first time series of data representing at least one locomotion characteristic from the inertial data;
applying dynamic time warping to the inertial data from said IMU sensor;
receiving a reference model template, the reference model template including a second time series of data representing the at least one locomotion characteristic for the locomotion category;
determining whether the first time series of data and the second time series of data match.
if the first and second time series of data match, activating a search for a position system signal; and if said position system signal is detected, initiating sampling position data from the positioning system module configured to be mounted to a user to obtain position information;
sending, in response at least in part to a determination of a match, an instruction to estimate a speed of the person according to said IMU data; and to update a corresponding speed model for the locomotion category by storing said instruction as a flag in a buffer for being read by a processor for updating said corresponding speed model and adjusting said speed model according to said position system signal and said estimated speed;
and
determining if a gait change has occurred according to a change in said corresponding speed model.

2. The method of claim 1, further comprising:
determining, using a positioning system module, the presence of a position system signal;
sampling position data from the positioning system module configured to be mounted to a user to obtain position information; and
receiving a third time series of the position data;
wherein the sending an instruction to update the corresponding speed model is in response at least in part to the determination of the presence of a position system signal.

3. The method of claim 2, wherein the instruction to update the corresponding speed model includes an instruction to update the speed model with speed data generated from the third time series of data.

4. The method of claim 3, wherein the determining whether the first time series of data and the second time series of data match includes applying a dynamic time warp vector to a plurality of data point pairs from the first time series data and the second time series data. The method of claim 2, wherein the positioning system is selected from the group consisting of GPS and a local positioning system.

6. The method of claim 1, further comprising: classifying a locomotion kinematics sequence to analyzing said gait; wherein said determining if said change in gait occurred is further performed according to said analysis of said gait.

7. The method of claim 6, wherein the determining whether the first time series of data and the second time series of data match includes determining if an average change between two corresponding indices of the two time series of data is less than 15%.

8. The method of claim 1, further comprising extracting parameters from said IMU data to determine biomechanical information of a gait of the person, wherein said parameters are selected from the group consisting of duration of movement, velocity, and IMU orientation in 3D space according to an analysis of geometric shape of the IMU signal at each cycle.

9. The method of claim 1, wherein said determining whether the first time series of data and the second time series of data match comprises determining whether locomotion of the person matches the locomotion category of said reference model template.

10. The method of claim 9, wherein if said locomotion of the person matches the locomotion category of said reference model template, a category of activity of the person is classified according to said reference model template.

11. The method of claim 10, wherein the determining whether the first time series of data and the second time series of data match, comprises determining said match if an average change of an index of the first time series of data to a matching index of the second time series of data, as a percentage of the index of the first time series of data, for each pair of indexes from the first and second time series of data, is less than 15%.
